(19) United States
(12) Patent Application Publication
(10) Pub. No.: US 2025/0058023 A1
(43) Pub. Date: Feb. 20, 2025

(54) SHAPED AMALGAMATED CARTILAGE GRAFTS AND METHODS FOR MAKING AND USING SAME

(71) Applicant: Musculoskeletal Transplant Foundation, Edison, NJ (US)

(72) Inventors: Marc Long, Monmouth Junction, NJ (US); Eric Semler, Morganville, NJ (US); Evangelia Chnari, Scotch Plains, NJ (US); Todd Nilsen, Thousand Oaks, CA (US); Roman Shikhanovich, Staten Island, NY (US); Jeffrey Cartmell, Freehold, NJ (US)

(21) Appl. No.: 18/807,214

(22) Filed: Aug. 16, 2024

Related U.S. Application Data
(60) Provisional application No. 63/532,931, filed on Aug. 16, 2023.

(51) Int. Cl. A61L 27/38

(52) U.S. Cl. CPC A61L 27/3847; A61L 2430/02

(57) ABSTRACT

Shaped amalgamated cartilage grafts having predetermined three-dimensional shapes are provided which are formed from a plurality or population of cartilage derived pieces (e.g., particles, fibers, fragments, etc.) by any one or more manufacturing and processing techniques including additive manufacturing (e.g., 3-D printing), stereolithography, molding, casting, extrusion, molding or shaping followed by dehydration, and any 3-D formation technique capable of converting the plurality of cartilage derived pieces into a shaped amalgamated cartilage graft having a predetermined 3-D shape which is maintained after hydration of the graft. Additional processing techniques and material properties may contribute to or increase cohesiveness of the plurality of cartilage particles to hold or maintain the predetermined 3-D shape including, inherent or enhanced entanglement of elongated cartilage particles or fibers, curing, cross-linking (thermal, chemical, UV, etc.), hydrostatic forces, surface tension, intermolecular forces, and combinations thereof.

### CROSS-REFERENCE TO RELATED APPLICATIONS

The present application claims the benefit of U.S. Provisional Application No. 63/532,931, filed Aug. 16, 2023, the entire disclosure of which is incorporated by reference herein.

### FIELD OF THE INVENTION

The invention described and contemplated herein relates to shaped amalgamated cartilage grafts and methods for making such grafts and using such grafts, especially in cosmetic and reconstructive surgeries.

### BACKGROUND

Current cosmetic and reconstructive surgical procedures which treat cartilaginous body features and regions, such as rhinoplasty procedures for treating noses, ear reconstruction, or body features and regions comprising cartilage and bone, sometimes involve recovery and formation of autologous monolithic cartilage grafts using cartilage recovered from the recipient undergoing the procedure, such as from a septum, an ear, a rib, etc., of the recipient. Use of such autologous cartilage grafts, of course, results in creation of an additional surgical site, which will also undergo healing with the attendant risk of infection and other complications. An additional issue sometimes encountered is the difficulty in recovering autologous cartilage of sufficient size and quantity to effectively treat the body feature which is the target of the surgical procedure.

Allogeneic cartilage grafts useful in cosmetic and reconstructive surgical procedures include monolithic costal cartilage, or monolithic strips or segments thereof, which are made from costal cartilage recovered, for example, from adult human cadaver donors. For example, unshaped and pre-cut costal cartilage grafts useful for use in rhinoplasty procedures are currently well known and available. Pre-cut costal cartilage allografts are commercially available under the tradename PROFILE® and EXTEARNA® Costal Cartilage Allografts (from MTF Biologics, located in Edison, New Jersey, U.S.A.) and are generally produced in a frozen form. Such cartilage allografts have distinct advantages to end users relative to uncut autograft and allograft cartilage segments. However, there are issues which limit the ultimate yield of such grafts producible from recovered costal cartilage including, for example, warping, cracking or both, of costal cartilage allografts during freezing, and the tendency of costal cartilage to become calcified as donor age increases. Both of these issues sometimes result in rejection of pre-cut costal cartilage allografts which are found to have warping, cracking, or undesirable calcification, which reduces the overall yield of useful grafts derived from donated allogeneic costal cartilage tissues. Additionally, a portion of the donated allogeneic cartilage tissue is sometimes unusable and, therefore, discarded and wasted, because it is insufficient in size or volume, or otherwise unsuitable (for example due to anatomically limited size, or having unsuitable straightness, curvature or contour), for making the desired cartilage grafts.

Either of the autologous or allogeneic cartilage grafts may be further trimmed and shaped at the time of surgery, to obtain grafts of appropriate and desired shape and size for the body feature being treated. Notably, when costal cartilage allografts are reshaped or resized at the time of surgery, there has been some unpredictability of the final size and shape of the graft. It is also known to place multiple cartilage pieces into a defect site to form a cartilage derived implant having the desired size and shape to fit the defect site in some surgical procedures.

Surgical practitioners and their patients would welcome the development of cartilage-derived grafts for use in cosmetic and reconstructive surgical procedures that are pre-shaped to have the desired size and shape for the body feature being treated, and would avoid issues with warping, cracking and partial calcification, as well as enabling use of smaller cartilage segments and pieces and, thereby, increase the graft yield per donor and maximize the gift of donation. It is believed that the shaped amalgamated cartilage grafts address the foregoing issues.

### SUMMARY

The invention described and contemplated herein relates to an amalgamated graft for treating a body feature or region comprising a treatment site, where the treatment site has a size, one or more dimensions, a volume, linearity, flatness, one or more curvatures, one or more contours, or a combination thereof, and the amalgamated graft comprises: a plurality of cartilage derived pieces which are amalgamated and formed into a predetermined three-dimensional shape which is retained during storage, after preparation, after implantation, or a combination thereof; and one or more biologically active substances, one or more additives, one or more binders, one or more carriers, one or more solvents, one or more non-cartilaginous materials, one or more tissue-derived matrices, one or more scaffolds, one or more mechanically supportive components, one or more types of cells, or a combination thereof. In some embodiments, the amalgamated cartilage graft may be lyophilized or hydrated in a storage solution. In some embodiments, the amalgamated cartilage graft may be capable of storage at temperatures above freezing for a period of storage time.

The predetermined three-dimensional shape of the graft is selected and has a size, one or more dimensions, a volume, linearity, flatness, one or more curvatures, one or more contours, or a combination thereof, which enable the graft to fit, conform to, or fit and conform to, the size, the one or more dimensions, the volume, the linearity, the flatness, the one or more curvatures, one or more contours, or a combination thereof, of the treatment site.

The graft is further capable of being reshaped to a subsequently selected three-dimensional shape, by manual manipulation, and then retaining that subsequently selected dimensional shape after reshaping, after implanting at a treatment site, or both after reshaping and implanting, wherein the subsequently selected three-dimensional shape has a size, one or more dimensions, a volume, linearity, flatness, one or more curvatures, one or more contours, or a combination thereof, which enable the graft to fit, conform to, or fit and conform to, the size, the one or more dimensions, the volume, the linearity, the flatness, the one or more curvatures, the one or more contours, or a combination thereof, of the treatment site.

### DETAILED DESCRIPTION

Shaped amalgamated cartilage grafts and methods for making and using them will now be described. The following terms have the following meanings throughout the following description, unless specifically stated otherwise.

As used herein, the term “amalgamated” means formed from or comprising a plurality of smaller pieces or components, such as cartilage pieces.

The terms “binder” and “binding agent” are used interchangeably herein and mean substances which hold ingredients, components and other substances in a composition or formulation together in a coherent mass, dosage form, or desired structure. Binders and binding agents include, for example but without limitation, saccharides (e.g., disaccharides, polysaccharides such as starch, cellulose and derivative thereof, etc.); natural polymers which are water soluble and can form hydrogels (e.g., collagen, alginate, gelatin, fibrinogen, hyaluronan, silk, agarose, chitosan, etc.); cellulose, gelatin; biodegradable synthetic polymers (e.g., poly(lactic-co-glycolic acid) (PLGA), polylactic acid (PLA), polyglycolide (PGA), polyurethane (PU), polycaprolactone (PCL), polyvinyl alcohol (PVA) and polyethylene glycol (PEG), etc.), non-biodegradable, biocompatible synthetic polymers such as polyether ether ketone (PEEK), poly(methyl methacrylate (PMMA), ultra high molecular weight polyethylene (UHMWPE), high modulus polyethylene (HMPE); biodegradable or non-biodegradable bioceramics such as tri-calcium phosphate (TCP), calcium sulfate, calcium phosphates and apatites, hydroxyapatite (HA), and combinations thereof.

The term “biocompatible” as used herein refers to causing no clinically relevant tissue irritation, injury, toxic reaction, or immunological reaction to living tissue.

The term “carrier” means a pharmaceutically acceptable inert agent or vehicle of any phase (e.g., solid, liquid, gel, etc., or combinations thereof) for delivering one or more active agents to a subject, and often is referred to as “diluent” or “excipient.” The carrier must be of sufficiently high purity and of sufficiently low toxicity to render it suitable for administration to the subject being treated. The carrier further should maintain, or at least not interfere with, the stability and bioavailability of an active agent and includes, for example without limitation, water, saline solution, cellulose and derivatives thereof, saccharides, mineral oil, gelatin, starch, polyethylene glycol, alcohols, and combinations thereof. Suitable carriers are typically selected based, at least in part, on the other materials or components with which they are combined or in contact, the type of tissue or body feature with which they will come in contact during use, and the route of delivery to a tissue or body feature. Several substances and materials suitable for use as carriers are also useful and suitable as binders, as can be seen from the definition provided above for “binder” and “binding agent.”

As used herein, the term “donor” includes any living or deceased individual of any species or class, including but not limited to mammalian (e.g., human, primate, bovine, porcine, ovine, equine, etc.), reptilian (lizard, turtle, crocodile, etc.), avian (ostrich, chicken, etc.), amphibian (frog, toad, salamander, etc.), all fishes (tuna, shark, trout, etc.) from which one or more tissue samples are harvested or recovered. Each donor may be one or more of: autologous (the same individual as the recipient of the tissue sample or graft produced therefrom), allogenic (the same species, but a different individual, as the recipient of the tissue sample or graft produced therefrom), and xenogenic (an individual of a different species as the recipient of the tissue sample or graft produced therefrom).

The term “graft,” as used herein, refers to a tissue or organ transplanted from a donor to a recipient. It includes, but is not limited to, a self-tissue transferred from one body site to another in the same individual (“autologous graft”), a tissue transferred between genetically identical individuals or sufficiently immunologically compatible to allow tissue transplant (“syngeneic graft”), a tissue transferred between genetically different members of the same species (“allogeneic graft” or “allograft”), and a tissue transferred between different species (“xenograft”).

The terms “resorption” and “resorbable” and their grammatical variants mean processes, and being susceptible or vulnerable to such processes, respectively, of breaking down or degrading a material or substance into its components and assimilation or elimination of the components, such as through biologic, physiologic, pathologic activity and processes.

The term “scaffold” means a material, substance, or device which provides physical or mechanical support or a substrate for supporting or carrying another material, substance, or device. For example, without limitation, a scaffold may be a structure capable of supporting a three-dimensional tissue formation. A three-dimensional scaffold is believed to be useful to replicate the in vivo milieu and to allow the cells to influence their own microenvironment. Scaffolds may serve to promote cell attachment and migration, to deliver and retain cells and biochemical factors, to enable diffusion of vital cell nutrients and expressed products, to exert certain mechanical and biological influences to modify the behavior of the cell phase, or any combination of these effects. Several features or characteristics are advantageous for a scaffold utilized for tissue reconstruction. For example, high porosity and an adequate pore size should facilitate cell seeding and diffusion of both cells and nutrients throughout the whole structure. Biodegradability of the scaffold may also be an important property. It is advantageous for a scaffold to be absorbable by the surrounding tissues without the necessity of a surgical removal, such that the rate at which degradation occurs coincides as closely as possible with the rate of new tissue formation. As cells are fabricating their own natural matrix structure around themselves, the scaffold would provide structural integrity within the body or body feature and eventually degrades leaving the neotissue (newly formed tissue) to assume the mechanical load.

Maintaining the predetermined three-dimensional shape, including but not limited to desired linearity, flatness, curvature, and contour, at a treatment site in a patient for a long period of time is desirable. For amalgamated grafts comprising a cartilage tissue-derived portion and at least one other component which is non-tissue-derived, the cartilage-tissue derived portion may remodel over time after implantation, but preferably without losing or changing that desired linearity, flatness, curvature, and contour of the predetermined three-dimensional shape. In some embodiments, for example without limitation, the amalgamated graft may include a non-tissue-derived component which is or contains at least one non-resorbable material or element which enables the graft to retain the predetermined three-dimensional shape and its linearity, flatness, curvature, and contours, during storage, preparation and implantation. In such embodiments, remodeling and resorption of the cartilage-tissue derived portion in vivo (i.e., during and after implantation at the treatment site) should occur without loss or destruction of desired linearity, flatness, curvature, and contour maintenance.

The shaped amalgamated cartilage grafts described and contemplated herein will now be described. The shaped amalgamated cartilage grafts generally comprise a plurality of cartilage pieces formed into a predetermined three-dimensional (3-D) shape by any of several effective manufacturing techniques, as described below. The shaped amalgamated cartilage grafts are capable of retaining their predetermined 3-D shape during storage, and upon preparation, rehydration, and implantation (i.e., without warping or losing its 3-D shape). An amalgamated cartilage-derived graft is considered to have warped or lost its predetermined 3-D shape if one or more of its size, dimensions, volume, linearity, flatness, curvature, and contours have changed to a degree where the graft no longer fits and conforms to an intended treatment site, or where a significant change in the predetermined 3-D shape occurs which is visible to the eye or with instrumentation and would, therefore, fail to provide the desired aesthetic outcome during reconstruction or repair of the body feature or region to be treated.

When the amalgamated cartilage-derived grafts are provided in a dehydrated (i.e., dried, lyophilized, etc.) state prior to use, they are capable of retaining their predetermined three-dimensional shape for a rehydrated period of time, which means the period of time from rehydration to use of the graft. The rehydrated period of time is from about 10 minutes to about 12 hours, including any period of time therebetween. Alternatively, when the amalgamated cartilage-derived grafts are provided in a hydrated (i.e., “pre-hydrated”) state prior to use, they are capable of retaining their predetermined three-dimensional shape for a pre-use period of time, which means the period of time from completion of production (or manufacture) to use of the graft. The pre-use period of time is from about 1 week to about 3 years, including any period of time therebetween.

After implantation, the amalgamated cartilage grafts are capable of retaining the predetermined three-dimensional shape for a post-implantation period of time, which means the period of time beginning with completion of implantation, with or without fixation, of the graft at a treatment site of a body feature or region being treated. The post-implantation period of time is from about 1 week to about 10 years, including any period of time therebetween which is required to achieve and maintain the desired size, dimensions, volume, linearity, flatness, curvature, contours, etc., of the body feature or region being treated.

The shaped amalgamated cartilage grafts are also capable of being reshaped to a subsequently selected 3-D shape by manual manipulation and then retaining that subsequently selected 3-D shape. The predetermined 3-D shape of the grafts is intentional (rather than simply incidental to a container, receptable, or other environmental structure) and selected to fit, conform, or be otherwise suitable for treatment of a particular body feature by cosmetic or reconstructive surgery. Furthermore, the shaped amalgamated cartilage grafts described and contemplated herein are not limited to use for treatment of cartilage tissue of body features or regions, but are also useful for treatment of bone, or both cartilage and bone present at body features or regions which would benefit from reconstructive or cosmetic procedures.

For example, without limitation, it is contemplated that the grafts described herein may be sized and shaped for use in treatment of a nose by rhinoplasty procedure, or for use in ear reconstruction and repair, laryngotracheal reconstruction and repair, craniomaxillofacial (CMF) augmentation, reconstruction, and repair, nipple reconstruction and repair, for use as joint interpositional spacers. Additional contemplated uses include, without limitation, treatment of a jaw by orthognathic reconstruction or repair, cranial reconstruction or repair, orthopedic (musculoskeletal) reconstruction or repair, and closure of defective or damaged cartilage and/or bone. The amalgamated cartilage-derived grafts described and contemplated herein would also be useful in treatment (reconstruction, repair, and regeneration) of an articular joint (e.g., knee, elbow, shoulder, hip, ankle, finger/toe, etc.), as well as condyle defect in an articular joint. As will be readily recognized by persons of ordinary skill in the relevant art, amalgamated cartilage-derived grafts would be useful for performing many other surgical procedures and treatments involving body features or regions which include cartilage, bone, or both, that would benefit from reconstruction and repair, whether for cosmetic or medical reasons.

The plurality of cartilage “pieces” includes, without limitation, fragments, particles, fibers (i.e., elongated particles or fragments), slices, segments, chunks, etc., and combinations thereof. The plurality of cartilage pieces may be produced from intact cartilage tissue, or obtained from another source (e.g., already formed or produced prior to formation of the grafts described and contemplated herein). In some embodiments, the plurality of cartilage pieces is produced by subjecting intact cartilage tissue recovered from one or more donors to any one or more size reducing techniques, with the resulting size-reduced cartilage pieces then amalgamated and shaped into a shaped amalgamated cartilage graft having a predetermined 3-D shape. The intact cartilage tissue and the resulting plurality of cartilage pieces may include one or more types of cartilage including, but not limited to, costal cartilage, articular cartilage, non-articular cartilage, fibrocartilage, and auricular cartilage. Other sources of cartilage-like tissue which can be utilized using the described invention include meniscus, intervertebral disc, annulus fibrosis, nucleus pulposus, and pubic symphysis.

The shaped amalgamated cartilage grafts described and contemplated herein are formed from the plurality of cartilage particles by any one or more manufacturing and processing techniques including, without limitation, additive manufacturing (e.g., 3-D printing), stereolithography, molding (with or without a mold having a predetermined 3-D shape), casting (with a mold having a predetermined 3-D shape), extrusion, molding or shaping followed by dehydration, and any 3-D formation technique capable of converting the plurality of cartilage pieces into a shaped amalgamated cartilage graft having a predetermined 3-D shape which is maintained upon preparation, rehydration, and implantation of the graft. Additional processing techniques and material properties may contribute to or increase cohesiveness of the plurality of cartilage particles to hold or maintain a desired 3-D shape including, without limitation, entanglement of elongated cartilage particles or fibers, curing, cross-linking (thermal, chemical, UV, etc.), hydrostatic forces, surface tension, intermolecular forces, and combinations thereof.

Additionally, it is contemplated that the grafts described herein may be produced as part of a personalized medical treatment method in which a patient's body feature or region which would benefit from treatment is scanned, surveyed, or mapped, to obtain information relating to the size, dimensions, volume, linearity, flatness, shape, contours, etc., of that feature or region, followed by forming a personalized shaped amalgamated cartilage graft having one or more of the size, dimensions, volume, linearity, flatness, shape, contours, etc., of the body feature or region to be treated. Such a customized graft will fit or conform closely to the size, dimensions, volume, linearity, flatness, shape, contours, etc., of the body feature or region into, onto, or proximate to, which it is implanted.

The shaped amalgamated cartilage grafts may be provided in any of several final forms including, without limitation, frozen, refrigerated, freeze-dried (lyophilized), capable of storage above freezing temperatures, capable of storage at below freezing temperatures, hydrated (i.e., only partially dehydrated or substantially not dehydrated), re-hydrated (i.e., hydrated by contact with a biocompatible hydration fluid after being at least partially dehydrated), etc.

The shaped amalgamated cartilage grafts formed from a plurality of cartilage pieces, reduce or avoid the aforementioned issues with warping, cracking, and partial calcification presented by conventional costal cartilage allografts. The shaped cartilage derived grafts also enable the use of smaller cartilage segments and pieces since they are processed into pieces and then formed into desired predetermined 3-D shapes. This ultimately increases the graft yield per donor and maximizes the gift of donation. This also provides more consistency in the grafts and ensures straightness and uniformity. Furthermore, the ability to precisely control microgeometry (e.g., surface roughness, texture, porosity, etc.) of the cartilage derived grafts during the forming process is also expected to impart additional advantageous features to the grafts.

In addition to retaining the predetermined 3-D shape, the shaped amalgamated cartilage grafts may be suturable (i.e., capable of substantially retaining at least one suture therein or therethrough without tearing or ripping such that the suture and graft are allowed to separate), as well as shapeable using cutting devices, while not being brittle. The shaped amalgamated cartilage grafts have mechanical properties similar to costal cartilage and known pre-cut costal cartilage allografts.

The shaped amalgamated cartilage grafts described and contemplated herein are slowly or minimally resorbable (over years), and preferably are substantially non-resorbable. The shaped amalgamated cartilage grafts present a cell-friendly scaffold or interface to surrounding host tissue (as opposed to being encapsulated in a fibrous matrix). These grafts may also encourage ingrowth from surrounding cells as to stabilize the graft following implantation and prevent graft migration (movement, shifting) over time.

The shaped amalgamated cartilage grafts described and contemplated herein may also include one or more additional biologically compatible materials, substances, or components, which may or may not be endogenous to cartilage tissue, including, but not limited to: biologically active substances, additives, binders, carriers, solvents, non-cartilaginous materials, tissue derived matrices, scaffolds or mechanically supportive components, one or more types of cells, and combinations thereof. The non-cartilaginous component(s) are biocompatible and may be used to impart long-term structural stability upon implantation. In some embodiments, non-cartilaginous materials or components may provide long-term structural stability to the shaped amalgamated cartilage grafts upon implantation.

The shaped amalgamated cartilage grafts should be engineered to have a prolonged, minimal, or even zero, resorption rate (i.e., rate at which the graft is resorbed and incorporated and/or replaced by the body's natural tissue at the implant site). The aforesaid prolonged, minimal, or zero, resorption rate is important because if the graft resorbs too fast, the intended and implemented architecture of the body part being treated (e.g., intended shape of a nose treated by rhinoplasty) will change detrimentally, and the treatment (reshaping or reconstruction) will fail. It is also important that the shaped amalgamated cartilage graft maintains appropriate surface properties, post-operatively, such that the graft is not encapsulated by fibrous tissue, but rather becomes biologically integrated with the surrounding host tissue to provide long-term implant positional stability and biocompatibility.

The shaped amalgamated cartilage grafts may comprise the above-described plurality of cartilage pieces as well as one or more biologically compatible binders (i.e., a “cartilage-binder graft”). Shaped amalgamated cartilage grafts may be manufactured having variances in porosity (and related biological properties) which allow for tailored incorporation (resorption) of different portions of the graft post-surgery. In some embodiments, the 3-D shape of such a cartilage-binder graft may comprise a simple block or brick shape. In some embodiments, the amalgamated cartilage-binder graft may comprise layers of cartilage and layers of non-cartilage materials, such as without limitation, a biocompatible polymer (e.g., either cartilage on the inside, or polymer on the inside). In some embodiments, the cartilage-binder graft may comprise a coating comprising costal cartilage pieces on the outside of a polymer scaffold. In some embodiments, the cartilage-binder graft may comprise composite sheets. In the composite sheets concept, costal cartilage may, for example, be cut into super-thin sheets, placed into a container, which is then filled with a binder, and then when the cartilage sheets and binder combination is cured, it may be cut to size and packaged. In some embodiment, cartilage particulates or fragments are mixed with a biopolymer or bioceramic to form a tissue: biomaterial composite and the desired 3D shape. For any and all such embodiments, the gradient of tissue: binder proportions may vary throughout the thickness of the shaped amalgamated cartilage graft. Other material properties may also be varied for the shaped amalgamated cartilage grafts which are malleable and reshapable during storage, while hydrated, after rehydration, or a combination thereof.

The shaped amalgamated cartilage grafts may include an array of holes, perforations, openings, slits, etc., to allow and facilitate cutting, as well as sutureability. Such holes, perforations, openings, slits, etc., may also allow acute connective tissue ingrowth so as to stabilize the graft in place post-surgery and, thereby, minimize or avoid undesired post-surgery movement or migration of the graft which could otherwise result in suboptimal results (e.g., deviation from intended linearity, contouring, or aesthetics). The holes, perforations, openings, slits, etc., may be provided at pre-determined intervals throughout the graft, or on one or more portions of the graft, or optionally only as pinholes on a periphery of the graft.

The 3-D shapes, features and geometries of the shaped amalgamated cartilage grafts may be designed for ease of implantation (drafts, wedges, etc.) or to prevent migration out of the surgical site (e.g., barbs, conical shape, etc.). The roughness of the surface of the shaped amalgamated cartilage grafts may also be controlled and designed during performance of the forming or manufacturing techniques to influence potential attachment of surrounding host cells, as well as incorporation or migration of the graft post-surgery.

In some embodiments, the shaped amalgamated cartilage grafts are capable of storage at room temperature (i.e., from about 18° C. to about 25° C.) for a period of storage time of at least about 72 hours. The grafts may be lyophilized, or hydrated in a storage solution. In some embodiments, the shaped amalgamated cartilage grafts are capable of storage at below freezing temperatures (i.e., about 0° C. or less). The grafts may be cryopreserved, with or without cryopreservation agents. In some embodiments, the shaped amalgamated cartilage grafts are substitutable and suitable for use as any other cartilage graft for both reconstructive (e.g., rhinoplasty post trauma, auricular reconstruction or Mohs procedures) and cosmetic surgical procedures to provide improved aesthetic outcomes. Examples of types of grafts used include dorsal overlay grafts, spreader grafts and columellar strut grafts.

In another embodiment, a shaped amalgamated cartilage graft may be formed by first producing a flowable bioink composition comprising a plurality of cartilage pieces, such as a plurality or population of cartilage particles, one or more binders, and one or more solvents and then using that flowable bioink composition to perform additive manufacturing (i.e., 3-D printing) to manufacture a graft having a printed 3-D shape.

Suitable sizes and dimensions of the cartilage pieces to be produced and then formed into 3-D shapes will vary depending upon factors including, but not limited to, the types processing to be applied to the cartilage particles, and the intended end use (e.g., which type of treatment or procedure of body feature will be involved). Selection of sizes and dimensions for cartilage pieces is generally within the understanding and skill of persons of ordinary skill in the relevant art. Nonetheless, when cartilage pieces are to be combined or formulated, as mentioned above, to produce a flowable bioink composition, suitable sizes of the cartilage particles (i.e., non-elongated particulate forms) include, without limitation, an average diameter of from about 5 microns (um) to about 1 millimeter (mm), such as less than about 500 μm. In some embodiments, a plurality or population of cartilage particles having average diameter of less than about 250 μm, or even less than 100 um, may be suitable or preferable for formulation into a bioink. It is noted that the narrower the size range of the cartilage particles which are produced, the more uniform the cartilage particles in that population will be which may further provide more uniformity during additive printing of the bioink and in the 3-D shape produced from those particles. Additional contemplated cartilage particle size ranges include, without limitation, from about 10 μm to about 100 μm, or from about 20 μm to about 200 μm, or even from about 20 μm to about 100 um.

The 3-D shape of the 3-D printed grafts may be simple, complex, or customized to conform to the intended surgical site. In some embodiments, the flowable bioink composition is delivered or extruded through the nozzle of a 3-D printer to create a specific predetermined 3-D shape with precisely controlled surface and bulk microstructure and porosity. Once the solvent evaporates, the binder will impart cohesivity to the 3-D amalgamated cartilage graft, which is then ready for implanting. Furthermore, the binder(s) are dispersed throughout such 3-D printed amalgamated cartilage grafts such that the cartilage pieces are held together in the predetermined 3-D shape, which remains intact during storage, preparation, rehydration, and implantation. In preferred embodiments, the shaped amalgamated cartilage graft maintains its 3-D shape for substantial periods of time post-implantation, maintaining the desired linearity, flatness, contour and shape.

Each of the one or more binders or binding agents may be resorbable, partially resorbable, or non-resorbable, and may comprise natural materials, synthetic materials, or a combination thereof. By selection of an appropriate binder or binding agent, which is generally within the ability of persons of ordinary skill in the relevant art, the amalgamated cartilage graft can be tailored so that resorbable and partially resorbable binders are resorbed at a predetermined, predictable rate, thus leaving behind a porous matrix (e.g., comprising a non-resorbable binder or non-resorbable portion of the binder) having a desired porosity. In some embodiments, the binding agents may comprise natural polymers such as collagen, gelatin, fibrinogen, hyaluronan, silk, agarose, chitosan that are water soluble and can form hydrogels. In other embodiments, the binding agents may comprise biodegradable synthetic polymers such as poly(lactic-co-glycolic acid) (PLGA), polylactic acid (PLA), polyglycolide (PGA), polyurethane (PU), polycaprolactone (PCL), and polyethylene glycol (PEG), or non biodegradable biopolymer such as polyether ether ketone (PEEK), poly(methyl methacrylate (PMMA), ultra high molecular weight polyethylene (UHMWPE), high modulus polyethylene (HMPE), or bioceramics such as calcium phosphates, apatites, or calcium sulfate.

In some embodiments, a mixture or combination of a plurality of cartilage pieces and one or more binders may be molded, manually or using a mold or other shaping technique, to form a shaped amalgamated cartilage graft having a 3-D shape. Such molded grafts comprising cartilage pieces and one or more binders also provide a 3-D structures in which the binder(s) are dispersed throughout the 3-D graft, whereby the cartilage pieces are held together to form the predetermined 3-D shape, which remains intact during storage, after preparation, and upon rehydration and implantation. In preferred embodiments, the shaped amalgamated cartilage graft maintains its 3-D shape for substantial periods of time post-implantation, maintaining the desired linearity, flatness, contour and shape.

Suitable ratios of cartilage pieces to binder include, without limitation, from about 25:75 to about 90:10. For example, additional suitable ratios of cartilage pieces to binder include about 70:30, or about 75:25, or about 80:20, or about 85:15. It should, of course, be understood that the ratio of cartilage pieces to binder may and will sometimes depend, at least in part, on the chemical characteristics and properties of the contemplated binders.

In addition, any of the above described embodiments of the shaped amalgamated cartilage grafts may further comprise a partial or full coating formed from tissue-derived slurries (e.g., costal cartilage) or secondary biomaterials (such as polyetheretherketone; PEEK). Such coatings may be formed using any known coating techniques, such as dip coating, spray coating, overlaying, painting, etc.

Furthermore, in some embodiments, at least a portion of a shaped amalgamated cartilage graft may be at least partially crosslinked. Such crosslinking is useful for adjusting and controlling the mechanical integrity, or the resorption rate, or both, of the final graft. More particularly, increased crosslinking will decrease the rate of resorption. Suitable cross-linking agents and techniques include, for example without limitation, aldehydes, EDC, genipin, ultraviolet (UV) light/radiation, radiation (e.g., Gamma, E-Beam, etc.), heat, etc. Crosslinking may also modify one or more mechanical properties of the graft. For example, increased crosslinking may provide greater structural integrity, increased stiffness, and greater resistance to deformation of the graft.

In some embodiments, a kit is provided that includes a cartilage powder (e.g., comprising fine cartilage particulates) or fragments/small shapes (larger particulates) and one or more binding agents which are either already combined together, or may be mixed together by a surgical practitioner, and then molded by a surgical practitioner during a surgical procedure. A measuring template (e.g., of the nose, in profile, for rhinoplasties) to assist the surgeon with measuring the nose may also be included in the kit to assist the surgical practitioner with the molding and shaping. Alternatively, simple shape templates, shaping tools, or other ancillary equipment to assist the surgical practitioner may be included in the kit.

Methods for producing the shaped amalgamated cartilage grafts described and contemplated hereinabove will now be described. Generally, such methods comprise the steps of: providing a plurality of cartilage particles, optionally combining or mixing the plurality of cartilage particles with one or more binders, optionally combining one or more additional biologically compatible materials, substances, or components with the plurality of cartilage particles, and forming the plurality of cartilage particles into a 3-D shape by performing one or more manufacturing techniques capable of effectively converting the plurality of cartilage pieces into a shaped amalgamated cartilage graft having a predetermined 3-D shape and wherein the graft is capable of maintaining the predetermined 3-D shape after hydration of the graft.

Suitable manufacturing techniques include, for example without limitation, additive manufacturing (e.g., 3-D printing), stereolithography, molding (with or without a mold having a predetermined 3-D shape), casting (with a mold having a predetermined 3-D shape), subtractive manufacturing (e.g., computer numerical control, or “CNC” devices and processes), and any 3-D formation technique capable of converting the plurality of cartilage pieces into a shaped amalgamated cartilage graft having a predetermined 3-D shape which is maintained after hydration of the graft.

The step of providing a plurality of cartilage pieces may comprise obtaining already formed cartilage pieces, or by producing a plurality of cartilage pieces from one or more intact cartilage tissue samples obtained from one or more donors. As previously described above, the plurality of cartilage pieces may comprise, without limitation, pieces, fragments, particles, fibers (i.e., elongated particles or fragments), slices, segments, etc., and combinations thereof. The plurality of cartilage pieces may be produced by any of several processes which include subjecting intact cartilage tissue recovered from one or more donors to any one or more size reducing techniques. Suitable size reducing techniques include, without limitation, one or more of freezer-milling, grating, slicing, shaving, grinding, cutting, and the like. Although costal cartilage is discussed extensively above, any type of allograft cartilage may be used including costal cartilage, articular cartilage, non-articular cartilage, fibrocartilage, hyaline cartilage, elastic cartilage, auricular cartilage, and combinations thereof. Other sources of cartilage-like tissue which can be utilized using the described invention include meniscus, intervertebral disc, annulus fibrosis, nucleus pulposus, and pubic symphysis.

In one embodiment, the manufacturing technique comprises a molding and reforming process in which cartilage particles are combined with at least one binding agent that imparts cohesivity between the particles. Alternatively, temperature and pressure may be used to impart cohesiveness or crosslinking may be performed.

In some embodiments, the method for producing a shaped amalgamated cartilage graft comprises mixing or otherwise combining a plurality of cartilage pieces with at least one binding agent and a solvent to create a flowable bioink composition suitable for forming a predetermined 3-D shape by 3-D printing with a 3-D printer. The predetermined 3-D shape may be simple, complex, or even a specific 3-D shape with precisely controlled surface and bulk microstructure and porosity. After allowing the solvent to evaporate, the binder imparts cohesivity to the resulting shaped amalgamated cartilage graft. Suitable binding agents are as previously described above.

The cartilage-binder mixture can be utilized as a 3D-printing “bioink”. Alternatively, the plurality of cartilage pieces may be combined with only one or more binders and then molded to form the graft having a 3-D structure.

The method for producing a shaped amalgamated cartilage graft may further comprise coating the graft with one or more tissue-derived slurries or additional materials or substances (e.g., PEEK), using any effective coating techniques, such as dip coating, spray coating, overlaying, painting, etc.

In some embodiments, the method for producing a shaped amalgamated cartilage graft may comprise forming a block from a tissue-binder combination (as described above) and applying an alternative manufacturing technique, such as subtractive manufacturing using a CNC milling device and process. CNC devices and processes are capable of forming intricate, precise shapes. Similarly, simple rectilinear 3-D shapes can also be milled.

The method for producing a shaped amalgamated cartilage graft may further comprise, after forming a 3-D shaped graft, the step of at least partially crosslinking at least a portion of the 3-D graft. Crosslinking may be performed using crosslinking agents and techniques such as, for example without limitation, aldehydes, EDC, genipin, ultraviolet (UV) light/radiation, radiation (e.g., Gamma, E-Beam, etc.), heat, etc. As previously explained, greater degrees of crosslinking may decrease the post-implantation resorption rate of the graft, increase structural integrity, increase stiffness, and increase resistance to post-implantation deformation of the graft. The resulting shaped amalgamated cartilage grafts are useful for performing cosmetic and reconstruction surgical procedures to treat body features, including but not limited to a nose (rhinoplasty), external ear (e.g., to correct microtia or anotia by ear reconstruction surgery), and facial skin after Mohs surgery to remove skin cancers. These grafts are useful for performing cosmetic and reconstructive resurfacing, planing or contouring, as well as other cosmetic and reconstruction surgical procedures requiring maintenance of tissue structure using an allograft-derived, non-resorbing/slow-resorbing graft, for example without limitation, 3-D nipple projection and/or reconstruction, gluteal augmentation, and abdominal muscle definition.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

1. (canceled)

2. An amalgamated graft for treating a body feature or region comprising a treatment site having a size, one or more dimensions, a volume, one or more curvatures, one or more contours, or a combination thereof, the amalgamated graft comprising a plurality of cartilage derived pieces which are amalgamated and formed into a predetermined three-dimensional shape which is retained after hydration, after implantation, or after both hydration and implantation.

3. The amalgamated graft of claim 2, wherein the predetermined three-dimensional shape of the graft is selected and has a size, one or more dimensions, a volume, one or more curvatures, one or more contours, or a combination thereof, which enable the graft to fit, conform to, or fit and conform to, the size, the one or more dimensions, the volume, the one or more curvatures, one or more contours, or a combination thereof, of the treatment site.

4. The amalgamated graft of claim 2, wherein the amalgamated cartilage graft is lyophilized or hydrated in a storage solution and capable of storage at room temperature for a period of storage time.

5. The amalgamated graft of claim 4, wherein the period of storage time is at least about 72 hours.

6. The graft of claim 2, wherein the graft is capable of being reshaped to a subsequently selected three-dimensional shape, by manual manipulation, and then retaining that subsequently selected dimensional shape after reshaping, after implanting at a treatment site, or both after reshaping and implanting.

7. The amalgamated graft of claim 6, wherein the subsequently selected three-dimensional shape has a size, one or more dimensions, a volume, one or more curvatures, one or more contours, or a combination thereof, which enable the graft to fit, conform to, or fit and conform to, the size, the one or more dimensions, the volume, the one or more curvatures, the one or more contours, or a combination thereof, of the treatment site.

8. The amalgamated graft of claim 2, further comprising: one or more biologically active substances, one or more additives, one or more binders, one or more carriers, one or more solvents, one or more non-cartilaginous materials, one or more tissue derived matrices, one or more scaffolds, one or more mechanically supportive components, one or more types of cells, and combinations thereof.

9. The amalgamated graft of claim 8, comprising one or more cartilage layers each comprising cartilage and one or more non-cartilage layers each comprising at least one binder, at least one carrier, at least one tissue derived material, at least one scaffold, at least one mechanically supportive component, or a combination thereof, wherein each of the one or more cartilage layers comprises amalgamated cartilage pieces or cartilage or a cartilage sheet, wherein the cartilage layers may alternate with the non-cartilage layers or not, and wherein the graft has a gradient of tissue: binder proportions which vary throughout the graft or not.

10. The amalgamated graft of claim 9, comprising a non-cartilage scaffold comprising a polymer and the cartilage layer comprising a coating of amalgamated cartilage pieces deposited on the non-cartilage scaffold.

11. The amalgamated graft of claim 9, comprising a plurality of cartilage layers each of which comprises a cartilage sheet and a plurality of non-cartilage layers each of which comprises a cured binder, wherein the cartilage layers alternate between the non-cartilage layers.

12. A method for treating a body feature or region comprising a treatment site having a size, one or more dimensions, a volume, one or more curvatures, one or more contours, or a combination thereof, the method comprising implanting the amalgamated graft of claim 1 into, onto, or proximate to the treatment site.

13. The method of claim 12, further comprising, prior to implanting the amalgamated graft: scanning, surveying, mapping, or a combination thereof, the treatment site to obtain information relating to the size, the one or more dimensions, the volume, the one or more curvatures, one or more contours, or a combination thereof, of the treatment site; and
selecting the predetermined three-dimensional shape to enable the amalgamated graft to fit, conform to, or fit and conform to, the size, the one or more dimensions, the volume, the one or more curvatures, one or more contours, or a combination thereof, of the treatment site.

14. The method of claim 12, wherein the treatment site comprises: a nose treatable by a rhinoplasty procedure; an ear treatable by a reconstruction procedure; a larynx, a trachea, or both a larynx and a trachea, treatable by a laryngotracheal reconstruction procedure; one or more craniomaxillofacial (CMF) bones treatable by an augmentation or reconstruction procedure, a nipple treatable by a reconstruction procedure, an articular joint having a condyle defect, or a joint treatable by an interpositional spacer implantation procedure.

15. A method for producing an amalgamated graft comprising a plurality of cartilage derived pieces which are amalgamated and formed into a predetermined three-dimensional shape, the method comprising the steps of:
providing a plurality of cartilage derived pieces;
optionally, combining or mixing the plurality of cartilage derived pieces with one or more binders;
optionally, combining or mixing the plurality of cartilage derived pieces with one or more carriers;
optionally, combining one or more additional biologically compatible materials, substances, or components with the plurality of cartilage derived pieces;
forming the plurality of cartilage pieces into a predetermined three-dimensional shape by performing one or more manufacturing techniques capable of effectively amalgamating the plurality of cartilage derived pieces into the amalgamated cartilage graft having a predetermined three-dimensional shape which is retained after hydration, after implantation, or after both hydration and implantation; and
optionally, at least partially crosslinking at least a portion of the amalgamated graft.

16. The method of claim 15, wherein the manufacturing techniques include: additive manufacturing, stereolithography, molding with or without a mold having the predetermined three-dimensional shape, casting with a mold having the predetermined three-dimensional shape, subtractive manufacturing.

17. The method of claim 15, wherein the step of providing a plurality of cartilage derived pieces comprises: obtaining already formed cartilage derived pieces, producing a plurality of cartilage derived pieces from one or more intact cartilage tissue samples obtained from one or more donor, or a combination thereof.

18. The method of claim 17, wherein the plurality of cartilage derived pieces comprises, pieces, fragments, particles, fibers, slices, segments, and combinations thereof.

19. The method of claim 15, therein the one or more manufacturing techniques comprise:
(A) a molding and reforming process in which the plurality of cartilage derived pieces are combined with at least one binding agent which imparts cohesivity between the cartilage derived pieces;
(B) applying temperature, pressure, or both, which impart cohesiveness; or
(C) a combination of (A) and (B).

20. The method of claim 15, further comprising either:
selecting a predetermined three-dimensional shape having features comprising a size, one or more dimensions, volume, one or more curvatures, one or more contours, or a combination thereof, which will enable the amalgamated graft to fit, conform to, or both fit and conform to corresponding features of a treatment site; or
selecting a subsequently selected dimensional shape having features comprising a size, one or more dimensions, volume, one or more curvatures, one or more contours, or a combination thereof, which will enable the amalgamated graft to fit, conform to, or both fit and conform to corresponding features of a treatment site.

21. The method of claim 15, wherein the amalgamated graft comprises a plurality of cartilage layers and a plurality of non-cartilage layers, and wherein:
the step of providing a plurality of cartilage derived pieces comprises providing or forming a plurality of cartilage sheets;
the step of combining the plurality of cartilage sheets with one or more binders is performed, wherein the one or more binders comprises at least one curable binder;
the step of forming the plurality of cartilage pieces into a predetermined three-dimensional shape is performed by at least the steps of:
placing the plurality of cartilage sheets in a container,
adding the one or more binders comprising at least one curable binder in a quantity sufficient to cover the plurality of cartilage sheets in the container,
curing the at least one curable binder with the plurality of cartilage sheets therein to produce a cured, layered cartilage-binder form; and
either using the cured, layered cartilage-binder form as the amalgamated graft having the predetermined three-dimensional shape, or reshaping the cured, layered cartilage-binder form by cutting, milling, shaving, or a combination thereof, to produce at least one amalgamated graft having a subsequently selected three-dimensional shape which is retained after hydration, after implantation, or after both hydration and implantation.
